(19) United States
(12) Patent Application Publication
(10) Pub. No.: US 2021/0262043 A1
(43) Pub. Date: Aug. 26, 2021

(54) CANCER SCREENING AND THERAPEUTICS

(71) Applicant: Nick Cyrus Arjomand, Santa Monica, CA (US)

(72) Inventor: Nick Cyrus Arjomand, Santa Monica, CA (US)

(21) Appl. No.: 17/316,494

(22) Filed: May 10, 2021

Related U.S. Application Data
(63) Continuation of application No. PCT/US2019/061021, filed on Nov. 12, 2019.
(60) Provisional application No. 62/759,996, filed on Nov. 12, 2018.

(51) Int. Cl. C12Q 1/6886

(52) U.S. Cl. CPC C12Q 1/6886; C12Q 2600/156

(57) ABSTRACT

The invention herein generally relates to early stage cancer screening and therapeutics, and particular methods of detecting and treating lung cancer. The invention identifies one or more mutations in genes, such as 15-HPGD. This can be done after collecting any suitable biological sample from the patient, such as their fingernail, saliva or blood. After collection, DNA is isolated/extracted and analyzed using a gene sequencing method. The gene sequencing method will detect predetermined one or more mutations inside this single gene's base pairs caused by the cancer, such as mutations created from an incremented drop in oxygen caused by the cancer cells (a bi-product mutation) and/or mutations that fail to suppress tumor growth for cancer. By blocking biological pathways associated with the bi-product mutation, the growth of the tumor can be reduced or prevented thereby improving prognosis.

### CROSS REFERENCE TO PRIOR APPLICATION

This application is a continuation of International Application No. PCT/US2019/061021, filed on Nov. 12, 2019, which claims priority and benefit under 35 U.S.C. 119(e) from U.S. Provisional Application Ser. No. 62/759,996, filed on Nov. 12, 2018, each of which is incorporated herein by reference.

### BACKGROUND

### 1. Technical Field

This invention relates generally to early stage (stage 1) cancer screening and therapeutics, such as for lung cancer. The invention identifies one or more mutation(s) in genes, such as 15-RPM (a bi-product mutation), as a diagnostic method. Gene mutations can be determined using any suitable method, such as gene sequencing DNA from a biological sample from the patient. Biological samples can be collected from any suitable source such as their fingernail, saliva, or blood. This invention is applicable for the identification of other bi-product mutations in order to gain data around the pathogenesis of malignant diseases. As used herein the term “bi-product mutation” relates to mutations that do not cause cancer, but rather are mutations that are caused by the hypoxic conditions cancer creates.

### 2. Background

Since the beginning of clinical medicine, Clubbed Fingernails (dubbed the Hippocrates Finger by the ancient Greeks) were the first known biomarker for an underlying disease. Clubbed Fingernails are caused by a mutation in the 15-HydroxProstaGlandin Dehydrogenase gene (HPGD;601688) on chromosome 4q34 in the fingernail which causes the enzyme PGHD to overproduce the ProstaGlandin PGE2, which in turn causes the tissue of all the nails to move at a 160 degree angle, curving the whole nail in a “clubbed” shape.

This condition of Clubbed Fingernails has been linked as an early warning call to a few types of pulmonary, gastric, and cardiac diseases, such as Crohn's Disease and tuberculosis, but this condition is usually (90-93 percent of the time) associated with lung cancer.[1] Unfortunate—about 97 percent of patients suffering from lung cancer do not show any physical symptoms (e.g., clubbed fingernails), and so their disease cannot be readily diagnosed by the naked eye. Thus, there is a need for a simple cancer screening and therapeutics, such as for lung cancer.

The teachings herein directed to using certain genes as biomarkers is a breakthrough in the field. While the prior art screens for mutations in genes that cause cancer, the tests described herein identifies “bi-product mutations” that are caused by early stage cancer, such as through hypoxic conditions, and uses those mutations as biomarkers for cancer screening. The methods described herein will jumpstart a new age in biomedical diagnostics, by targeting the world's most deadliest cancers in order to treat said cancers, preferably early on.

### DETAILED DESCRIPTION OF THE INVENTION

The present application is directed to cancer screening and therapeutic methods revolving around the identification of one or more silent mutations in a patient's DNA, collected from any suitable biological sample. The identification of said mutations can be used as a biomarker in detecting cancer, preferably in an early stage. In one example, this application is directed to an early stage lung cancer screening method that identifies one or more silent mutations in the homozygous gene 15-HPGD. The one or more silent mutations can be identified from any suitable biological sample from the patient, such as DNA extracted from the nail, spit or blood, for example. Any suitable gene identification method, such as gene sequencing, can be used with the teachings herein.

Recent research in the area of the sensitivity of genes and silent mutations has suggested (in other cancers/diseases such as Lyme's Disease and HIV) that, although the phenotypical expression of Clubbed Fingernails itself may not be present as a visible warning, the gene 15-HPGD (which causes nail clubbing) could still mutate (more as the tumor grows larger), usually due to a slight/somewhat drop of oxygen in the blood (not dangerous, but also not normal) caused by the appearance of an early stage lung cancer tumor. But, due to the genomic structure and behavior of most people, and/or because the drop of oxygen in the blood is usually not enough to cause the mutation to overproduce the ProstaGlandin PGE2 (making it have no effect on the phenotype), the actual condition of would not appear, and the person would not contract nail clubbing, classifying it as a silent mutation caused by an early stage tumor.[2] In addition, it is noted that because the density of a tumor cell is greater than a regular cell, malignant tumor cells dividing in the lung take up more oxygen. This problem is compounded because cancer cells are also pressing into the lung during stage 1 lung cancer. Consequently, the amount of oxygen in and the flow of oxygenated blood into and out of the lungs decreases, causing specific incremented mutations in the 15-HPGD gene. More specifically, the hypoxic situation increases as the mutation spreads and gets more effectual. Additionally, many other genes are affected by this drop in oxygen, and this allows the tumor micro environment to grow in strength by weakening and incrementally unraveling these genes. In addition, research cited herein establishes that patients that display fingernail clubbing are carriers of a specific autoimmune disease, which provides evidential support that the one or more mutations occur more often. Whatever the specific biological pathway, the one or more mutations in 15-HGPD are biomarkers for early stage lung cancer detection. The data cited herein supports that the disclosed methods are applicable not only for lung cancer but for all types of cancer and other diseases.

Data Relating to Use of Nail DNA

A research project done by J PreMed Public Health (2012) inquiring about possible bio-markers in the nail's DNA, tested 12 subject's nails for strong DNA amplification. The paper then concluded that, based on the results of the subject's DNA, amplification seemed very promising, and that testing/identifying biomarkers in the nail's DNA was a plausible diagnostic for many diseases, including some cancer.[3]

A separate research project performed by Mot Biotech (2010), tested the proteinase of the Cucumis Melon to obtain a higher amplification of DNA extraction from the nail (3× higher than other proteinases). This paper concluded that the nail's DNA could be used for medical diagnostics including, detecting biomarkers.[4] A few more research projects revolving around this subject, have stated in their papers about the promising possibility of biomarkers in the nail's DNA.[5],[6]

1. Data Relating to Oxygen Linked Mutations

In a the research paper entitled “Diminished DNA Repair and Elevated Mutagenesis in Mammalian Cells Exposed to Hypoxia and Low pH” [7], the study showed that DNA in mammalian cells tended to diminish their DNA repair and that there was elevated mutagenesis in a wide variety of genes when exposed to certain levels of hypoxia and low pH, catalyzed by the tumor micro environment. The results of this study provide significant data that the tumor micro environment can cause an elevated rate of mutations in a wide variety of genes and in addition diminished DNA repair to correct these mutations (through hypoxia and low pH). This establishes that mutations in genes, such as 15-HPGD, are likely due to the loss of oxygen catalyzed by the tumor micro environment. See also [11].

The 15-HPGD gene has been associated as a cancer related gene by the human protein atlas (www.proteinatlas.org/ENSG00000164120-HPGD/pathology). In a research paper entitled, “Focusing Downstream in Lung Cancer Prevention: 15-Hydroxyprostaglandin Dehydrogenase,” [10] the authors stated: “The catabolic enzyme 15-hydroxyprostaglandin dehydrogenase (15-PGDH), recently identified to have tumor suppressor activity (28, 29), converts PGE2 to inactive 15-keto derivatives (30). Although the 15-keto derivatives are not active in mediating EP receptor-dependent signaling, they have recently been reported to have peroxisome proliferator-activated receptor-γ ligand activity and therefore could promote antitumor responses (31). Preventing the expression of 15-PGDH limits the degradation of PGE2, allowing it to accumulate and thus encouraging tumor growth.”

The eventual cell/genetic hypoxia that would occur is believed to be due to the accumulation of growing mutations caused by cell/genomic asphyxiation in growing tumors. There is a potential disruption by its spread (concluded through multiple reference points of data provided by NIH), particularly in more oxygen sensitive genes such as hydro genes.[7],[8] [9]. According to NCBI, a malignant tumor is defined by the consumption of oxygen, evidencing that the continual/methodical loss of oxygen is caused by the growth of the tumor, especially when comparing to data on patients with these genetic abnormalities.[9]

In sum, based on the data, there is sufficient evidence that enlarging tumor cells consume a higher oxygen yield than regular cells, thereby causing a subject's genome, particularly the 15-HPGD gene, to have a higher incidence of specific mutations. The resulting mutations trigger an autoimmune signal, in carriers of the autoimmune diseases PHO (pulmonary hypertrophic osteoarthropy), causing the occurrence of nail clubbing in this situation, and releasing hypoxic like deflation in the genome. Additionally, this would potentially allow the tumor micro-environment to strengthen its growth, as the gene grows weaker. It should be emphasized that the present invention is not limited to lung cancer or 15-HPGD.

2. Sequencing

### EXAMPLE 1

Whole genome sequencing/multi-gene sequencing (via liquid biopsy) will be used to identify specific bi-product mutations that provide insightful evidence of the pathogenesis of the disease and appropriate treatment (suggestive use of hydro genes). Patients who have confirmed/unconfirmed cases of a multitude of different stage diseases (notably certain types of cancer, such as lung cancer) and/or present a high risk, will be asked to submit a liquid biopsy (either nail, blood or spit) to their treatment provider who will then take the sample(s) to an appropriate facility. The facility can perform the sequencing, which will ultimately provide data regarding 15-HPGD gene mutations. This diagnosis will lead to a customized therapeutic regimen.

At the laboratory, basic protocol for whole genome sequencing/multi polypeptide sequencing using any accurate sequencing methods/identification methods will be optimized in order to produce the complete list of nucleotides in the human genome (likely through BAC Negative Strand). From there, the DNA will then be analyzed using common DNA analyzation protocol (via computer) to see which “bi-product mutations” have occurred in which genes. These results will facilitate a provider in understanding how the disease is affecting the human genome and which treatments will be the most effective at treating the disease. Given the sequencing's compatibility with common AI analyzation methods for diseases, it can be used to efficiently gain data from a multitude of points, in order to best understand the pathogenesis of the diseases. In addition, this test will be repeated multiple times, in order to better track the pathogenesis of the disease, and gather more data on points to target.

Based on the current research, the identification of one or more silent mutations can be used as a biomarker in earlier cancer stages thus acting as an effective cancer diagnostic and/or a supplement to a common biopsy. The diagnostic methods described herein can be utilized at first in high risk patients (smokers, and people with a cancer genetic bank) and then expanded into further development to lower risk patients. In addition, whole genome sequencing of early stage cancers, such as through nail or liquid biopsy, can be based on the one or more mutations described herein.

3. Clinical Trial/Analysis

### EXAMPLE 2

At least one hundred patients diagnosed currently with Stage 1 and 2 of non small cell lung cancer (preferably 50 percent of each stage and 50 percent of each gender and chosen randomly outside of this criteria, for a pretrial), who have not undergone any treatment vet, will provide a single average sized sample, such as a nail clipping, saliva or blood sample, to the lab that will perform the Analysis/Clinical trial. From there, the sample will be preserved. Subsequently, lab workers will then extract the DNA from the samples (e.g., nails) using any suitable method, such as a keratin degradation method, basic DNA extraction method, and any gene sequencing method. One or more silent mutations in the 15-HPGD gene on chromosome 4q34, or any other abnormalities/mutations inside the gene in 90 percent or higher of the patients (comparing the gene to (1-100) people confirmed NOT to have any form lung cancer), to prove that the test is successful. Multiple tests will be performed, and the same test will be performed on different types of lung cancer/stages of lung cancer. In addition, a potential “false positive” test, a test identifying the appearance of a silent mutation in the 15-HPGD gene of 100 people who have been confirmed not to have lung cancer, will be performed as well.

4. Potential Diagnostic/Analyzation Risk Test

### EXAMPLE 3

The following provides an example of how the diagnostic methods herein can be administered. A test to detect and/or analyze risk of early stages of lung cancer can be administered by local pharmacies and/or physicians. Alternatively, the test can be done at home and the biological samples, such as nail clippings, can be mailed to a laboratory for analysis. The test can be administered every 9-12 months for lower risk patients. The test can be administered every 1-6 months for higher risk patients. Higher risk patients would be patients who have one or more of the following criteria, for example: currently smoke or have formerly smoked, live in highly polluted areas, have a family history of lung cancer and/or who are over 49. This is merely one definition of “patients” and other criteria can be used. For example, test could be administered every 3-12 months for patients, above 18, who are currently smoking, have smoked in the past, been exposed to radon gas/other toxic chemicals, heavy exposure to secondhand smoke/heavy air pollution, have a family history of lung cancer/smoking, or are just wondering if they have a higher risk of obtaining the tumor.

The test involves the patient submitting a biological sample such as a nail clipping, a saliva swab, or blood, that will be sent to their local/closest approved laboratory via a pharmacy, physician, or mail. Once it reaches the lab, the lab workers will then extract the DNA from the nail by using any common degradation method/substance for the protein Keratin, 50 ul-100 ul of proteinase k solution), or any basic DNA extraction method depending on the biological sample submitted. From there, scientists will identify the gene 15-HPGD in chromosome 4q34 using any type of any gene sequencing methods (e.g., the Illumina Individual MiSeq Method) to detect specific mutations inside this single gene's base pairs caused by the cancer (because of the specific reaction to the drop of oxygen caused by the cancer cells). This analysis will determine if there are any mutations or abnormal defects inside the gene (bi-product mutations), and can take about 1-7 days. After the detection of a mutation/abnormality (or lack thereof), the patient's insurance provider will be notified and, if necessary the patient can be referred to an oncologist for a more extensive/detailed test and evaluation for either an elevated risk of lung cancer or lung cancer itself. This test could also be used for other cancers and diseases.

Additional Therapeutic Applications

In addition, the application can be expanded to a therapeutic setting. In the above pathway, the tumor creates a hypoxic environment which leads to a mutation in 15-HPGD which in turn limits the gene's suppression of the tumor. By blocking that pathway, the growth of the tumor can be reduced or prevented thereby improving prognosis. This could be used as a therapeutic tool, in order to help patients fight the growth of the disease and allow them to be treated properly and effectively through applications of such as CRSIPR, oral injection, drug development, and oxygen therapy. Any suitable cancer or disease treatments can be used with the methods herein, non-exclusively including surgery, chemotherapy, radiation, bone marrow transplant, immunotherapy, hormone therapy, targeted drug therapy, cryoablation, radiofrequency ablation, etc.

### CITATIONS

The above-referenced citations are provided below and each citation is incorporated by by reference herein in their entireties.

[1], [2] Digital Clubbing, Malay Sarkar, et al., Lung India, 2012 October-December:29(4): 354-362 doi [10.4103/0970-211.102824]

[3] Nail DNA and Possible Biomarkers: A Pilot Study, Joshua Park et al., J. Prev. Med. Public Health, 2012 July:45(4): 235-243, published online 2012 Jul. 31 doi [10.3961/jpmph2012.45.4.235]

[4] Efficient DNA extraction from nail clippings using the protease solution from Cucumis melo., Yoshida-Yamamoto S. et at., Mol. Biotechnology, 2010 September 46(1):41-8 doi [10.1007/s12033-010-9273-6.]

[5] DNA from Nails for Genetic Analyses in Large-Scale Epidemiologic Studies, Janneke G. F. Hogervorst et al., Cancer Epidemiology Biomarkers & Prevention, Vol, 23, issue 12, December 2014.

[6] High-quality DNA from Fingernails for Genetic Analysis, Sandra Preuner et al., The Journal of Molecular Diagnostics, Vol. 16, Issue 4, July 2014, Pages 459-466

[7] “Diminished DNA Repair and Elevated Mutagenesis in Mammalian Cells Exposed to Hypoxia and Low pH.” Jianling Yuan, Latha Narayanan, Sara Rockwell, and Peter M. Glazer, Volume 60, Issue 16, August 2000.

[8] The induced prostaglandin E2 pathway is a key regulator of the respiratory response to infection and hypoxia in neonates, Annika O. Hofstetter, Sipra Saha, Veronica Siljehav, Per-Johan Jakobsson, and Eric Herlenius PNAS Jun. 5, 2007 104 (23) 9894-9899; https://doi.org/10.1073/pnas.0611468104

[9] Hypoxia/Reoxygenation-Induced Mutations in Mammalian Cells Detected by the Flow Cytometry Mutation Assay and Characterized by Mutant Spectrum, Stephen B. Keysar, a Nadira Trncic, Susan M. LaRue, and Michael H. Fox Radiat Res. 2010 January; 173(1): 21-26. doi: 10.1667/RR1838.1

[10] “Focusing Downstream in Lung Cancer Prevention: 15-Hydroxyprostaglandin Dehydogenase,” Steven M. Dubinett, Jenny T. Mao, and Saswati Hazra, Doi 10.1158/1940-6207.CAPR-08-0148, September 20.

[11] “Genetic instability and the tumor microenvironment: towards the concept of microenvironment-induced mutagenesis,” Bindra R S and Glazer P M, Mutat. Res., 2005 January 6; 569(1-2):75-85.

I claim:

1. A method for screening cancer comprising:
a) providing a subject having a high risk for a type of cancer;
b) obtaining a biological sample that contains DNA from said subject;
c) extracting and isolating DNA from said biological sample;
d) analyzing the isolated DNA for the presence of a predetermined silent mutation that is linked to said type of cancer.

2. The method of claim 1, wherein the type of cancer is lung cancer.

3. The method of claim 2, wherein the predetermined silent mutation is present in the 15-HPGD gene.

4. The method of claim 3, wherein the patient does not have the physical appearance of clubbed nails.

5. The method of claim 3, wherein the biological sample is selected from the group consisting of: a nail clipping, blood, and saliva.

6. The method of claim 3, wherein the biological sample is a nail clipping.

7. The method of claim 6, wherein the DNA extraction comprises keratin degradation.

8. The method of claim 7, wherein a proteinase k solution is used for keratin degradation.

9. The method of claim 1, wherein the silent mutation is a bi-product mutation caused by hypoxic conditions from the cancer.

10. The method of claim 1, wherein the screening is done during stage 1 of the cancer.

11. The method of claim 4, wherein the silent mutation is a bi-product mutation caused by hypoxic conditions from the cancer.

12. The method of claim 1, wherein the silent mutation that is linked to said type of cancer is present; and further comprising administering a therapy regimen to said subject that is sufficient to treat or ameliorate said type of cancer.

13. The method of claim 12, wherein said therapy regimen is configured to correct the silent mutation or a biological pathway resulting from said silent mutation.

14. The method of claim 12, wherein said therapy regimen is selected from the group consisting of: surgery, chemotherapy, radiation, bone marrow transplant, immunotherapy, hormone therapy, targeted drug therapy, cryoablation, and radiofrequency ablation.

15. The method of claim 12, wherein said therapy regimen comprises oxygen therapy.
